(19) United States
(12) Patent Application Publication
(10) Pub. No.: US 2008/0175918 A1
(43) Pub. Date: Jul. 24, 2008

(54) CHEMICALLY CROSS-LINKED ELASTOMERIC MICROCAPSULES

(76) Inventor: Bryan E. Laulicht, (US)

Correspondence Address: BAKER BOTTS L.L.P., 30 ROCKEFELLER PLAZA, 44TH FLOOR, NEW YORK, NY, 10112-4498, US

(21) Appl. No.: 11/971,507

(22) Filed: Jan. 9, 2008

Related U.S. Application Data
(63) Continuation of application No. PCT/US2006/027163, filed on Jul. 12, 2006.
(60) Provisional application No. 60/699,412, filed on Jul. 13, 2005.

(51) Int. Cl. A61K 9/50; A61P 31/00; A61P 35/00

(52) U.S. Cl. 424/490

(57) ABSTRACT

The present invention relates to the fields of stably encapsulating oral care, skin care, scented, flavoring agents for cued release, and therapeutic agents for extended and sustained release. The invention relates to the stable microencapsulation of these agents for incorporation into dentifrices, topical ointments, microwavable food products, dryer sheets and chewing gums to be released during brushing, applying, heating, tumbling, and masticating respectively. Additionally, the invention encompasses extended and sustained release formulations that achieve reservoir-type delivery of therapeutic agents. The invention discloses methods for manufacturing and post-processing populations of chemically cross-linked elastomeric microcapsules allowing for the incorporation of encapsulated agents into a wide range of formulations without significantly altering their physio-chemical properties while providing for the cued delivery of the encapsulated agent upon the reception of a single or multiple mechanical or thermo-mechanical cues, or extended delivery via diffusion.

### RELATED APPLICATIONS

This application claims priority to U.S. Provisional Application No. 60/699,412, filed Jul. 13, 2005, the disclosure of which is incorporated herein by reference in its entirety.

### FIELD OF THE INVENTION

The present invention relates to the fields of stable encapsulated oral care, skin care, scented, and flavoring agents for cued release, and therapeutic agents for extended and sustained release. The invention relates to the stable microencapsulation of these agents for incorporation into dentifrices, topical ointments, microwavable food products, dryer sheets and chewing gums to be released during brushing, applying, heating, tumbling, and masticating, respectively. Additionally, the invention encompasses extended and sustained release formulations that achieve reservoir-type delivery of therapeutic agents.

### BACKGROUND OF THE INVENTION

Numerous microencapsulation techniques have been developed for a wide array of applications. The current liposome-based micro-encapsulation technologies however lack in providing for microcapsules that have control over geometry, specifically with regard to capsule wall thickness and capsule diameter (Gregoriadis Nature 1980, 283:814-815). To improve membrane strength with respect to liposomal technologies, amphiphilic di-block copolymers of poly(ethylene oxide)-poly(butadiene) have been synthesized and self-assembled into polymersomes owing to their amphiphilic nature. After self-assembly, the poly(butadiene) portion of the polymersome shell has been chemically crosslinked to yield increased mechanical strength. However, limitations similar to those of liposomes regarding controllability and range of wall-thickness, as well as capsule wall permeability, remain (Discher et al., Science, 1999, 284:1143-1146).

Additionally, the chemical and mechanical properties of the phospholipid bilayers of liposomes and shells formed by precipitation made in other emulsion technologies are governed only by weak electrostatic and hydrophobic forces. Thus, there remains a need for covalently bonded microcapsules affording them a greater range of mechanical stability and the ability to be incorporated into a greater range of chemical environments (e.g., environments containing surfactants and/or strong oxidizing agents).

Current technologies utilizing solvent evaporation use physically associated thermoplastic capsule walls, which have been shown to possess large pore sizes relative to the chemically cross-linked elastomeric microcapsules (Cohen et al., Pharmaceutical Research 1991, 8:713-720). The present invention addresses the need to afford the capability of making chemically cross-linked polymer microcapsules that are water-impermeable. As a result, the permeation of therapeutic agents releases slowly through the shells of chemically cross-linked elastomeric microcapsules relative to permeation through physically associated thermoplastic capsule shells enabling extended release formulations.

### SUMMARY OF THE INVENTION

The present invention comprises the first method for utilizing a multi-component encapsulant phase to form chemically-crosslinked elastomeric microcapsules in a controlled fashion.

The present invention provides a novel means of encapsulating solutions, dispersions, or suspensions in chemically cross-linked polymer microcapsules. In certain embodiments of the invention, delivery is achieved by means of mechanical rupture, by thermo-mechanical rupture, or by diffusion. Microcapsules comprising encapsulated fluids and chemically-crosslinked elastomeric shells provided for in the invention are spherical in shape upon their production and range in size from on the order of from about 2.5 to about 2,500 microns in diameter. Populations of microcapsules in the present invention allow for cued or sustained delivery of the encapsulated active agents. By varying the physical and mechanical properties of populations of microcapsules both during and after production, the present invention provides for the encapsulation of any aqueous solution or suspension of active agents within polymer microcapsules that are designed to rupture under predetermined mechanical or thermo-mechanical conditions in certain specific embodiments. Other embodiments provide for the encapsulation of apolar, oleophilic solutions relative to an encapsulant phase, solid particle suspensions or dispersions, or water-in-oil emulsions. In other embodiments, the shell remains intact for the duration of the delivery of diffusion-based release. Varying manufacturing process parameters systematically establishes shell thickness ranges and geometries from which to choose the mean and distribution of rupture strengths and active agent permeability of each population of microcapsules allowing for tailored release properties of the encapsulated media.

A manufacturing process for the present invention allows for the production and post-processing of populations of microcapsules to achieve controllable geometries, mechanical properties, and release kinetics. In certain embodiments, each population of microcapsules is manufactured to deliver its contents upon encountering mechanical stresses equaling or exceeding their rupture strength. By combining multiple populations of microcapsules, a single batch can utilize multiple mechanical cues to provide pulsatile or sustained release of the active agent. In specific embodiments the microcapsule wall thickness can be altered to achieve the desired release properties.

### DESCRIPTION OF THE DRAWINGS

FIG. 1. A photomicrograph (100× magnification) of unilamellar spherical microcapsules comprising organosiloxane polymer shells and aqueous cores.

FIG. 2. A photomicrograph (100× magnification) of a polymer microcapsule, depicted in FIG. 1, after mechanically-induced rupture.

FIG. 3. A photomicrograph (100× magnification) of multi-core polymer microcapsules.

FIG. 4. A photomicrograph (100× magnification) of polymer microcapsules formed without utilizing a carrier solvent resulting in thick-walled microcapsules.

FIG. 5. A photomicrograph (200× magnification) of polymer microcapsules formed utilizing a carrier solvent resulting in thin-walled microcapsules.

FIG. 6. Summary of gas chromatograph data showing 0.00 parts per million (PPM) of the carrier solvent (methylene chloride in this embodiment) remains after solvent evaporation. Data was independently verified by Scientech Laboratories, Inc. using United States Pharmacopeias method 467-I.

FIG. 7. An atomic force topological micrograph of an organosiloxane polymer film processed under the conditions of the present invention showing that the root mean squared surface roughness of the resultant film is 0.498 nanometers indicating the negligible pore size of the processed film as would be expected in the native organosiloxane film.

FIGS. 8(a)-(d). Graphical representations of nuclear reaction depth profiling data acquired before and after simulating one year of immersion in room temperature distilled water and concentrated aqueous hydrogen peroxide. The data demonstrate the negligibility of the differences in the hydrogen content at any given depth within the organosiloxane polymer film processed under the same conditions. Certain embodiments of the invention demonstrate the absence of permeation of the polymer by hydrogen containing molecules (i.e. water or hydrogen peroxide) during the simulated year of immersion.

FIG. 9. A photomicrograph (100× magnification) of organosiloxane polymer microcapsules containing an aqueous solution of hydrogen peroxide as provided for in a specific embodiment of the invention.

FIGS. 10(a)-(d). Graphical representations of Rutherford backscattering data acquired before and after simulating one year of immersion in room temperature distilled water and concentrated aqueous hydrogen peroxide compared with the simulated expected data yielded from intrinsic properties of the organosiloxane polymer processed in the same manner as the shells of the microcapsules provided for in a specific embodiment of the invention. Relative peak heights at the resonance frequencies specific to elemental oxygen and silicon remain constant throughout experimentation demonstrating the chemical inertness and impermeability of the polymer to one year of immersion in a strongly oxidizing environment.

FIG. 11. A photomicrograph (100× magnification) of spherical polymer microcapsules containing an aqueous solution of surface active cetyl pyridinium chloride evidencing the decreased mean capsule size in the presence of decreased interfacial tension.

FIG. 12. A photomicrograph (40× magnification) of spherical polymer microcapsules containing an aqueous solution of cetyl pyridinium chloride and sorbitol demonstrating the increased mean capsule size in the presence of increased viscosity of the encapsulated solution.

FIG. 13. A photomicrograph (100× magnification) of dimpled polymer microcapsules containing a low osmolarity aqueous sucrose solution surrounded by a high osmolarity sucrose solution demonstrating the morphological effects of an osmolarity imbalance.

FIG. 14. A photomicrograph (200× magnification) of a spherical polymer microcapsule surrounded by a high osmolarity sucrose solution, containing a higher osmolarity aqueous sucrose solution demonstrating the return to spherical morphology upon reversing the direction of the osmotic pressure gradient.

FIG. 15. A photomicrograph (200× magnification) of a multi-core polymer microcapsule containing a dispersion of solid sodium percarbonate particles suspended in light mineral oil.

FIG. 16. A photomicrograph (40× magnification) of single-core polymer microcapsules containing apolar (e.g., mineral) oil.

FIGS. 17(a)-(c). A series of photomicrographs (200× magnification) depicting a population of chemically cross-linked elastomeric microcapsules suspended in soybean oil prior to exposure to microwave radiation and after thermo-mechanical rupture due to exposure for 30 seconds and two minutes in a standard microwave oven. In Particular:

FIG. 17 (a) Population of Chemically Cross-linked Elastomeric Microcapsules Containing Water Suspended in Soybean Oil

FIG. 17 (b) Population of Chemically Cross-linked Elastomeric Microcapsules Containing Water Suspended in Soybean Oil after 30 Seconds in a Microwave Oven

FIG. 17 (c) Population of Chemically Cross-linked Elastomeric Microcapsules Containing Water Suspended in Soybean Oil after 2 Minutes in a Microwave Oven

FIGS. 18(a)-(c). A series of photomicrographs (200× magnification) depicting a population of rhodamine B-containing chemically cross-linked elastomeric microcapsules one hour and one year after manufacturing. Photomicrographs demonstrate rhodamine release into the surrounding aqueous media from the aqueous cores of chemically cross-linked polymeric microcapsules unstirred at room temperature for one year. Rhodamine levels within the microcapsules remain visible after the duration of one year indicating the capability of prolonged release of various therapeutic agents. Additionally, confocal microscopy shows that rhodamine is present within the elastomeric shell after one year further evidencing prolonged release of the model active. In Particular:

FIG. 18 (a) A Population of Rhodamine-containing Chemically Cross-linked Elastomeric Microcapsules 1 Hour after Production

FIG. 18 (b) A Population of Rhodamine-containing Chemically Cross-linked Elastomeric Microcapsules 1 Year after Production

FIG. 18 (c) Confocal Micrograph of Rhodamine-containing Chemically Cross-linked Elastomeric Microcapsules 1 Year after Production after Transfer to Rhodamine-depleted Aqueous Media Showing the Presence of Rhodamine in the Elastomeric Shells

FIG. 19. A photomicrograph (100× magnification) of an evaporatively dried population of chemically cross-linked elastomeric microcapsules.

FIG. 20. A photomicrograph (100× magnification) of a population of chemically cross-linked elastomeric microcapsules after sieving.

FIG. 21. A photomicrograph (100× magnification) of a population of chemically cross-linked elastomeric microcapsules after separation by density centrifugation.

### DETAILED DESCRIPTION

The present invention provides a technology for the formation of chemically cross-linked elastomeric microcapsules that allow for the physical separation of their contents from the ambient environment by an elastomeric shell that serves as a diffusion barrier for shell-permeable actives. The elastomeric shell remains intact until such time that sufficient stress is applied to rupture each individual microcapsule to release its contents.

The present invention involves a confluence of four distinct achievements. First is the development of a process that allows for the production of chemically cross-linked elastomeric microcapsule populations of a controllable mean diameter.

Second, the invention provides for a novel mechanism for forming and controlling the mean thickness of a polymer shell by utilizing a multi-component encapsulant phase that in certain specific embodiments contains pre-polymer, a cross-linking agent, and a carrier solvent, such that the entire encapsulant phase is immiscible with the encapsulated phase. Yet, the carrier solvent can be removed by the process of solvent evaporation (Kita et al., Nippon Kagaku Kaishi 1978, 1:11-14) prior to the chemical cross-linking of the elastomeric shells. To adjust shell thickness, the carrier solvent to elastomer ratio, mixing rates, and/or the polymer to active agent solution volume ratio can be varied systematically. Since the carrier solvent can be used to modify the viscosity and miscibility properties of the polymer encapsulant, the shells of the microcapsules can be composed of a wide range of biocompatible and orally acceptable polymers affording the microcapsule shells the ability to withstand corrosive contents (e.g., those containing strong oxidizing agents) and to reside in chemically diverse environments (e.g., those containing humectants and/or detergents).

The third achievement is the development of a system for controlling the elastic modulus of the capsule walls. To control the elastic modulus of the capsule walls, the pre-polymer and cross-linking agent molecular weights and compositions are varied.

Finally, the fourth achievement of the present invention permits the definition of the upper and lower bounds of the encapsulant volume fraction and the osmolarity of encapsulated media within the polymer microcapsule and the total microcapsule volume to increase uniformity of the final product by sieving, density separation, and osmolarity altering techniques during post-processing without hindering the scalability of the manufacturing process.

The various aspects of the invention will be set forth in greater detail in the following sections. This organization into various sections is intended to facilitate understanding the invention, and is no way intended to be limiting thereof.

### Definitions

“Shell-permeable” as used herein refers to an active agent that can, with sufficient time, diffuse across the elastomeric shell. “Shell-impermeable” as used herein reefers to a polymer shell that prevents at least ninety percent, more preferably greater than ninety-five percent, and most preferably greater than ninety-nine percent of the encapsulated active agent(s) from being introduced into the surroundings until it is ruptured.

The term “pre-polymer” as used herein refers to monomeric and oligomeric molecules that increase in effective polymer chain length upon vulcanization of curing into an elastomer. The term “polymer” as used herein refers to a molecule containing a plurality of covalently attached monomer units. The term polymer also includes branched, dendrimeric, linear, and star polymers as well as both homopolymers and copolymers.

The term “elastomer” as used herein refers to any polymer of an elastic nature.

The term “microcapsule” is used in this application to mean a spherical or nearly spherical structure ranging in diameter from on the order of about 2.5 to about 2,500 microns composed of a distinct polymer shell surrounding encapsulated media.

The term “population” is used in this application to mean a collection or group of microcapsules. The population can result from a single batch process or from a combination of groups from different batch processes.

The term “chemically cross-linked” in any of its grammatical forms used in conjunction with a polymer, refers to any covalent linkage of monomers or oligomers to form polymers.

The term “shell” or “wall” refers to the polymer component of the microcapsules surrounding the encapsulated media.

The term “multi-core” refers to microcapsules containing multiple cores within a single, spherical microcapsule separated by the polymer shell material both from the ambient environment, as well as from other fluid-containing cores.

The terms “curing agent” or “vulcanizing agent” refers to any molecular species that increases the effective chain length of monomeric and/or oligomeric units to form a chemically cross-linked polymer.

As used herein, the term “organic solvent” is intended to mean any carbon-based liquid solvent, preferably one that is immiscible with water in certain embodiments and preferably one that is immiscible with apolar oils in other embodiments when mixed with pre-polymer and curing agent components. Exemplary organic solvents include methylene chloride, ethyl lactate, ethyl acetate, chloroform, alcohols, and mixtures thereof.

The term “carrier solvent” means any organic solvent initially combined incorporated into the polymer containing phase that does not remain in the final product.

The terms “biocompatible” and “orally acceptable” refer to molecular entities, at particular concentrations, and compositions that are physiologically tolerable and do not typically produce an allergic or similar untoward reaction, such as gastric upset, fever, dizziness and the like, when used in the appropriate fashion by a human.

A “formulation” refers to the specific chemical and mechanical conditions necessary to achieve the desired population of microcapsules.

The terms “extended release,” “prolonged release,” and “sustained release” in any their grammatical forms as used herein refer to diffusion-based release of an active agent for greater than one month in some embodiments, more preferably three months, and most preferably one year.

The term “reservoir” as used herein in any of its grammatical forms refers to a type of therapeutic agent delivery system in which a quantity of an active agent is separated from its site of delivery by an therapeutic-agent-permeable membrane.

The term “solvent evaporation” refers to the process by which the carrier solvent evaporates quickly through the final immiscible phase during rapid agitation or mixing.

The term “emulsion stabilizer” refers to a class of amphipathic molecules that can align themselves at hydrophilic/hydrophobic or polar/apolar interfaces in such a manner as to reduce the interfacial tension and therefore increase the stability of an emulsion by reducing the energy necessary to maintain the interfaces between the suspended droplets in their surroundings.

The term “emulsion” refers to a suspension of one solution or suspension in another in which it is immiscible, in some applications in the presence of an emulsion stabilizer.

The term “water-in-oil” refers to any emulsion in which a more hydrophilic, in certain embodiments, or apolar, in other embodiments, solution or suspension is encapsulated in a more hydrophobic, in certain embodiments, or more polar, in other embodiments, encapsulant phase.

The term “oil-in-water” refers to any emulsion in which a more oleophilic, in some embodiments, and apolar, in other embodiments, solution or suspension is encapsulated in a more hydrophilic, in certain embodiments, or more polar, in other embodiments, phase. The term “water-in-oil-in-water” refers to any double emulsion in which the encapsulated phase is more hydrophilic in some embodiments, or more apolar in other embodiments, in nature than the hydrophobic or more apolar encapsulant phase. Additionally, the term indicates that two emulsions are being formed, the first is an initial hydrophilic, in certain embodiments, or more apolar, in other embodiments, phase suspended in an immiscible encapsulant phase, which is then introduced into a final hydrophilic phase to arrive at the initial, or encapsulated, phase surrounded by the immiscible encapsulant phase suspended in the final hydrophilic phase. In the case of hydrogen peroxide containing organosiloxane microcapsules, the initial “water” phase is the aqueous hydrogen peroxide solution containing a small amount of an emulsion stabilizer, the initial “oil” phase is the organosiloxane pre-polymer/curing agent in their carrier solvent, and the final “water” phase is an aqueous polyvinyl alcohol solution. In the oil microencapsulation embodiments the initial “water” phase is an apolar oil solution or suspension, while the other phases remain unchanged.

The term “thermo-mechanical” in any of its grammatical forms as used herein refers to thermal energy derived in any way (e.g. via the heating of water by radiation in the microwave range) that results in mechanical force.

The term “rupture strength” refers to the force required to break the microcapsule wall normalized by the cross-sectional area upon which the force is acting.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as an oral care formulation. For example “about” can mean within one or more than one standard deviations of the mean, per the practice in the art. Alternatively, “about” can mean a range within five-fold, and more preferably within two-fold, of a value. Where particular values are described in the application and claims, unless otherwise stated the term “about” meaning within an acceptable error range for the particular value should be assumed.

### Microcapsules

In embodiments of the present invention, chemically cross-linked elastomeric microcapsules range in diameter from about 2.5 to about 2,500 microns with polymer shells ranging from single to hundreds of microns, preferably with a range of diameters between about 10 to about 250 microns with shell thicknesses on the order of single microns for certain embodiments. As one of ordinary skill in the art would readily understand, volumes such as these are subject to measurement error, which in turn depends on how the measurement is made.

Within the cores of the microcapsules reside hydrophilic or apolar solutions or suspensions containing active ingredients to be delivered to the area of interest upon receiving the appropriate mechanical or thermal cue. Towards that end, in certain embodiments, microcapsules have been formulated with polymer shells that are impermeable to and chemically unaffected by the media they encapsulate even at very small shell thicknesses. As a result, the thickness and materials properties of the shells can be modified to achieve release under the desired mechanical conditions without affecting the permeability or chemical resilience of the polymer shell.

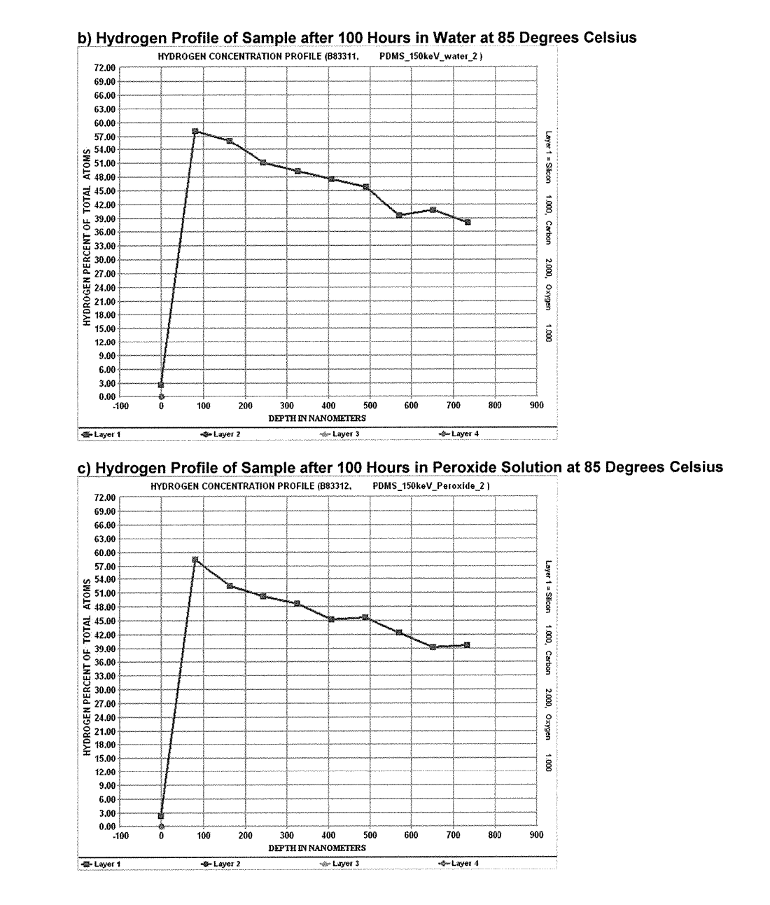

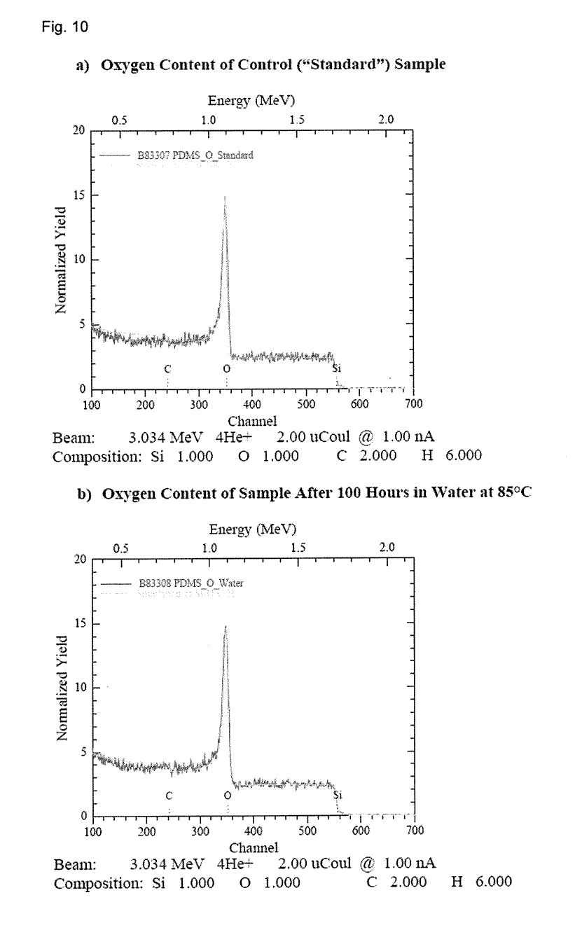

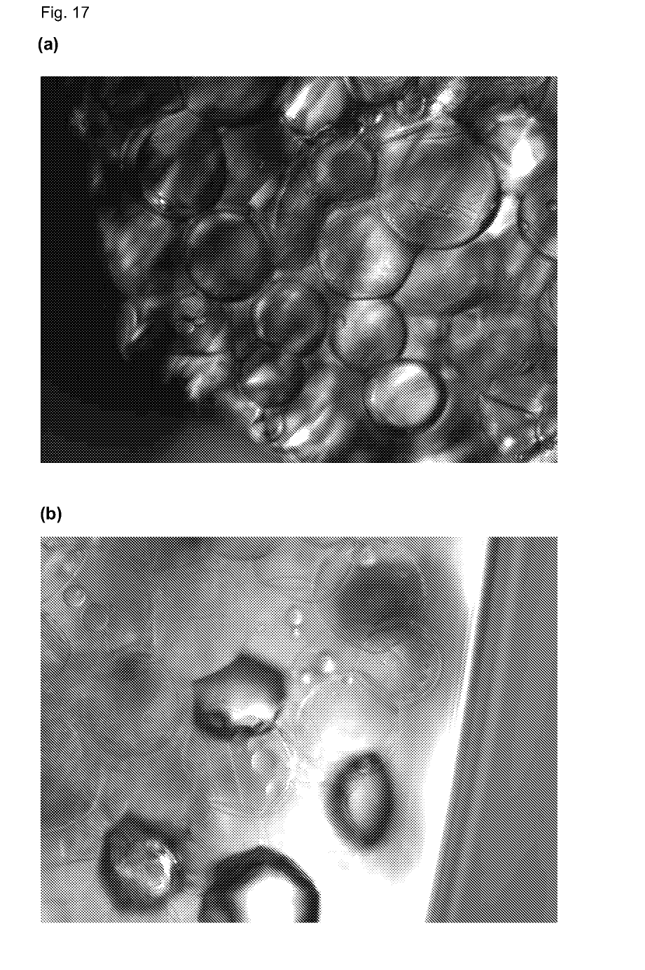

Although in certain embodiments the microcapsule shells are impermeable to the fluid contents, in some embodiments of the invention, the shells are permeable to gases (e.g., shells made from organosiloxane polymer). In those embodiments, the slow evolution of gas from the encapsulated liquid (e.g., oxygen liberated from hydrogen peroxide) will permeate the membrane into the surroundings without rupturing the capsule walls allowing for long term storage of volatile agents within the microcapsule cores while maintaining the mechanical integrity of the polymer shells as demonstrated by stability studies monitoring hydrogen and oxygen concentrations within the polymer shell material by nuclear reaction depth profiling and Rutherford backscattering (see FIGS. 8 and 10, respectively). Therefore, the mechanical and thermo-mechanical stresses necessary to rupture the microcapsules in the long term are not greatly affected during prolonged storage indicating a long shelf life. Although slow evolution of gas has been demonstrated not to compromise the barrier properties of the capsule shells, upon rapid heating of water-containing capsules by microwave radiation, thermally-cued rupture has also been demonstrated as shown in FIG. 17.

### Microcapsule Production

The manufacturing of the microcapsules in the present invention involves a two step process. In the first step, in certain embodiments, a water-in-oil emulsion of droplets of the hydrophilic or apolar phase is suspended in an immiscible encapsulant phase. In some embodiments, the encapsulated phase is stabilized by a small quantity of an appropriate emulsion stabilizer. To form the first emulsion in certain embodiments, a volume of the hydrophilic or apolar encapsulated phase is vigorously mixed with a larger volume of an immiscible, multi-component encapsulant phase containing the pre-polymer and curing agent dissolved in a carrier solvent. In other embodiments the encapsulated phase itself is an emulsion or solid particle suspension.

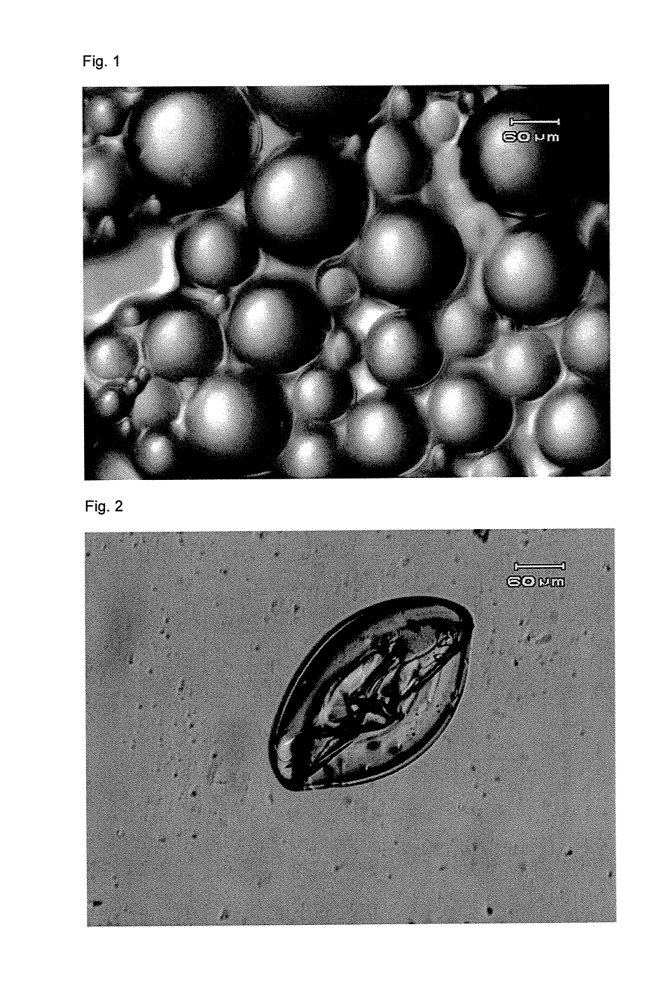

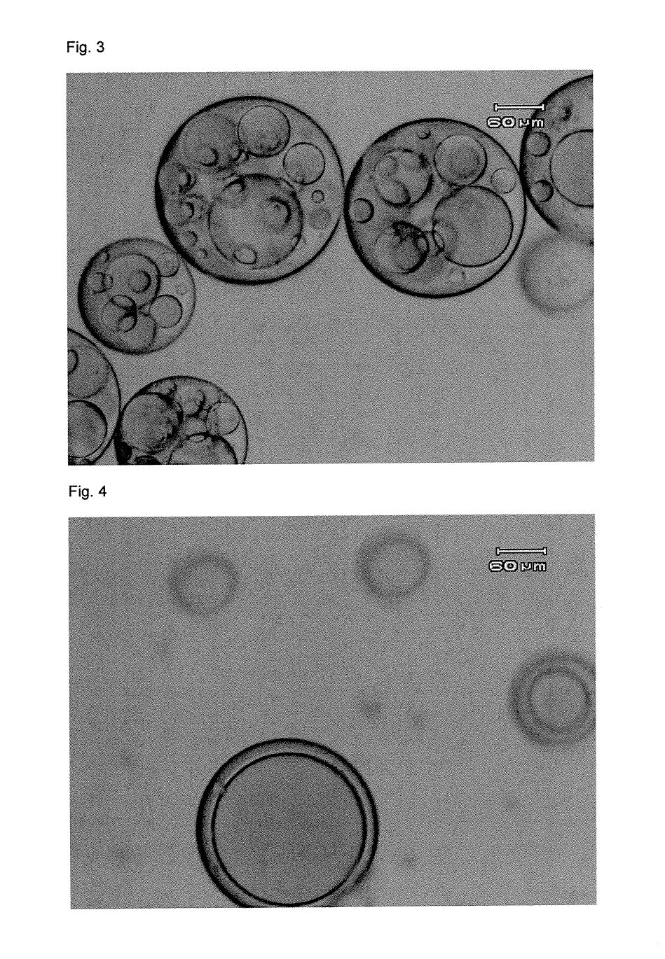

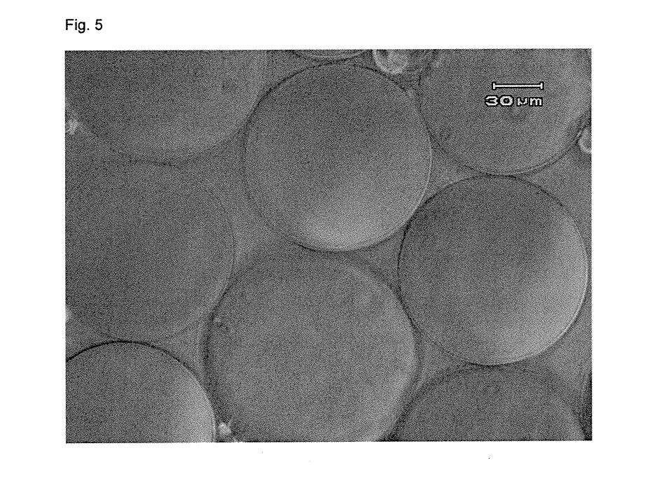

In the second step, in certain embodiments of the invention, the water-in-oil emulsion is suspended in a larger volume of an aqueous phase by mixing in the presence of an emulsion stabilizer to form a water-in-oil-in-water double emulsion. During the formation of the water-in-oil-in-water double emulsion, the carrier solvent in the hydrophobic phase evaporates via solvent evaporation leaving behind only the pre-polymer and curing agent in the encapsulant phase at which time the capsule shells cure to become a thermoset elastomer. The absence of carrier solvent is demonstrated by gas chromatography results that show the complete lack of the carrier solvent used in specific embodiments of the invention after processing. In certain embodiments of the invention, mechanical stirring of the double emulsion in the presence of an emulsion stabilizer is continued until the polymer shell has cured at a temperature commensurate with the thermal stability of the encapsulated active agent(s), the desired cure time, and the materials properties of the polymer. Once the polymer has cured, at final there are a large number of either, or a combination of both, unilamellar, (FIG. 1) or multi-core (FIG. 3), polymer microcapsules containing the encapsulated aqueous phase, or in other embodiments apolar oil phase, and a number of smaller, solid polymer microspheres all suspended in the final aqueous phase. Multi-core micro capsules form when the curing occurs prior to coalescence of the encapsulated phase, whereas unilamellar microcapsules form when the droplets have coalesced into a single reservoir prior to curing. After the polymer has cured, the population of resultant microcapsules is post-processed to remove remaining emulsion stabilizer, separate fluid core microcapsules from solid microspheres, and even further homogenize the resultant population by volume and volume fraction limiting as desired.

In certain embodiments of the invention, the initial aqueous or apolar oil phase is the phase that ultimately resides in the lumen of the microcapsules and is therefore the encapsulated phase, which may be comprised of any one or a combination of solutions, emulsions, or solid particle suspensions. In these embodiments, the initial aqueous, apolar oil, or encapsulated phase consists of the active agent being encapsulated either alone or in suspension or solution. To aid in the stabilization of the first emulsion process, minute amounts of emulsion stabilizer are added in certain embodiments. Also, in certain embodiments of the invention, using low concentrations of the emulsion stabilizer, as in the cases without an emulsion stabilizer, allows for distinct droplets of the initial aqueous phase to coalesce more quickly than in the presence of larger amounts of emulsion stabilizer the tending towards forming unilamellar microcapsules. In other embodiments of the invention, the emulsion is sufficiently stable and the polymer cures sufficiently quickly such that the droplets of the initial aqueous phase do not coalesce within the polymer containing phase yielding a population of multi-core microcapsules.

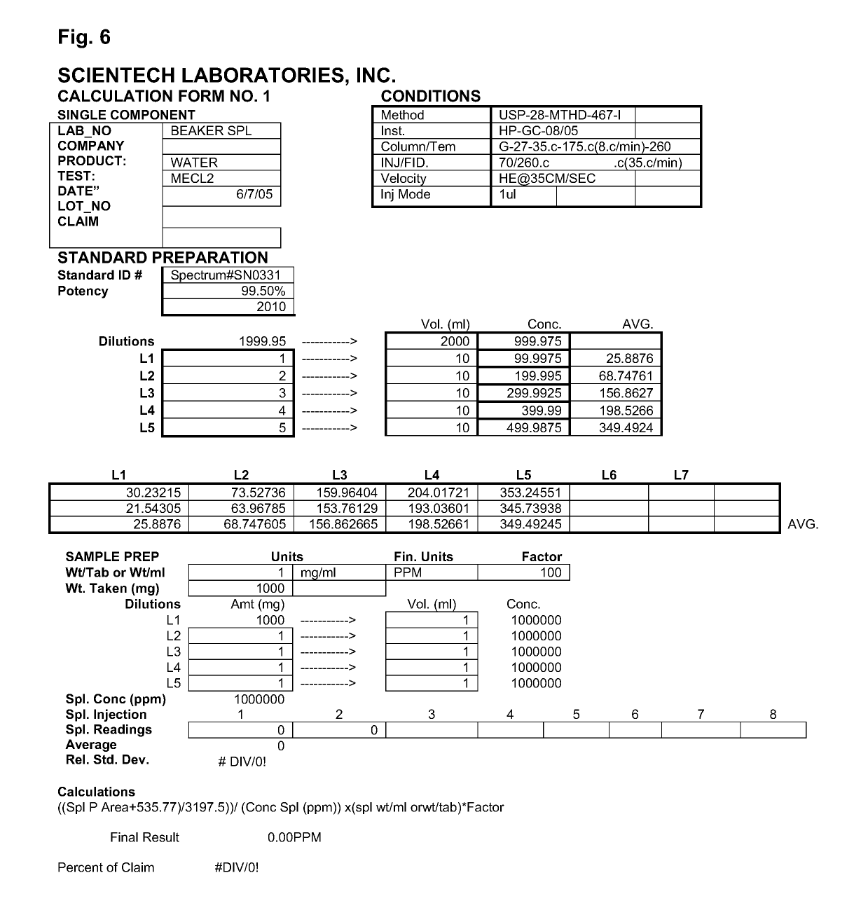

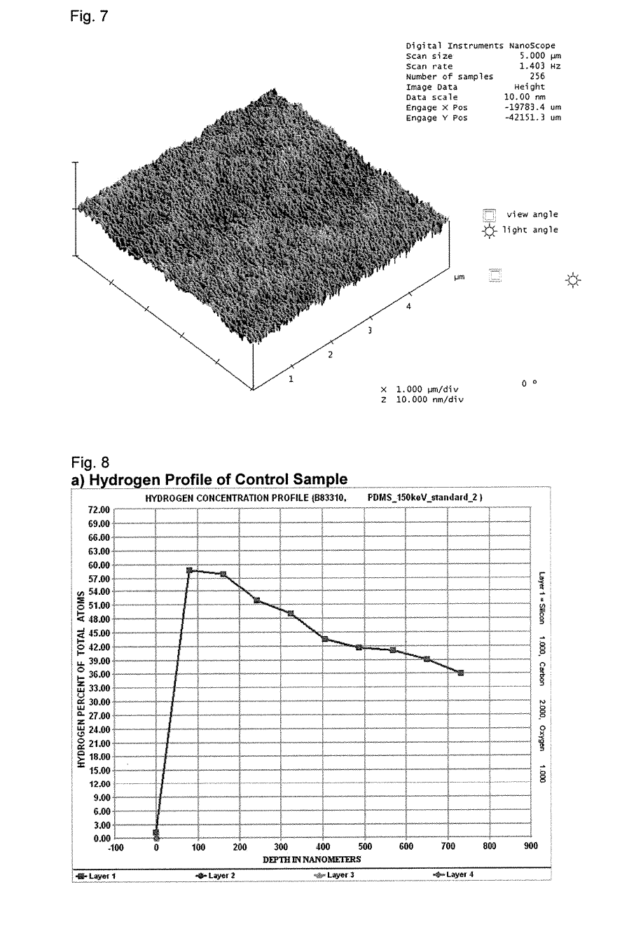

For embodiments of the invention in which a water-in-oil-in-water double emulsion is formed, the first emulsion is formed when the initial aqueous, or apolar oil, phase is suspended in the oil phase consisting of the uncured polymer, which may be a single or multi-component fluid of the pre-polymer and its cross-linking agent, and in some embodiments in solution with a carrier solvent that is effectively immiscible with the initial encapsulated phase(s). The volume of the encapsulant phase must exceed that of the encapsulated phase so as to avoid forming an oil-in-water emulsion. In the cases utilizing a volatile organic compound as the carrier solvent when biocompatibility, or oral acceptability, of the resultant microcapsules is desirable, it has been demonstrated that the volatile organic compound does not remain in the population microcapsules in any appreciable amount as tested by the methods set forth by the United States Pharmacopeias. Data are provided from a specific embodiment in FIG. 6.

Additionally, in the cases where a carrier solvent is used, it is important that the carrier solvent does not chemically alter the pre-polymer, its curing agent, or the resultant polymer significantly, and that the solvent be removed via solvent evaporation during processing before the polymer shell has cured at the desired processing temperature. Therefore, a carrier solvent for the specific embodiment of hydrogen peroxide must have a high vapor pressure below the temperature at which the hydrogen peroxide solution rapidly dissociates. In specific embodiments of the invention utilizing a volatile carrier solvent, the vigorous stirring, and large volume ratio of the final aqueous phase to the oil phase, combine to enable rapid solvent evaporation to take place. Additionally, the emulsion stabilizer contained in the final aqueous phase serves to prevent agglomeration of the microcapsules during the solvent evaporation and curing processes.

The percent yield of the encapsulation process is a measurement of the amount of the initial aqueous that is located within the capsule walls at final, or conversely the percentage of the initial aqueous, or apolar oil, phase that is not released into the final aqueous phase during processing. Factors such as the stirring or mixing conditions, the amount of emulsion stabilizer used in both emulsions, and the physical and materials properties of the solutions used in making the double emulsion will to some extent affect the percent yield of the final process.

### Methods for Adjusting the Mechanical Properties of the Microcapsule Walls

Shell thickness of the resultant microcapsules can be controlled by varying any one, or a combination of, processing conditions. In some embodiments of the invention, the constitution of the encapsulant phase can be altered to vary shell thickness (FIGS. 3 and 4). In the cases involving a carrier solvent, increasing the polymer to carrier solvent volume ratio results in increasing shell thickness and the tendency towards multi-core microcapsules. Additionally, increasing stirring rate and/or duration of either of the emulsifying steps would decrease the resultant droplet size of the suspension, which in the first emulsifying step works to decrease the inner diameter of the inner lumen of the microcapsule, and in the second emulsifying step works to decrease the outer diameter of the microcapsule thereby providing other control points for varying shell thickness. Finally, the greater the ratio of the volume of the polymer containing phase to the volume of the active containing phase the thicker the resultant polymer capsule walls holding the composition of the encapsulant phase constant. By being able to adjust wall thickness, the population of microcapsules can be tuned to rupture under well-defined conditions of mechanical stresses. Additionally, wall thickness control imparts therapeutic agent permeability in those embodiments in which chemically cross-linked elastomeric microcapsules contain permeable actives.

### Microcapsule Post-Processing

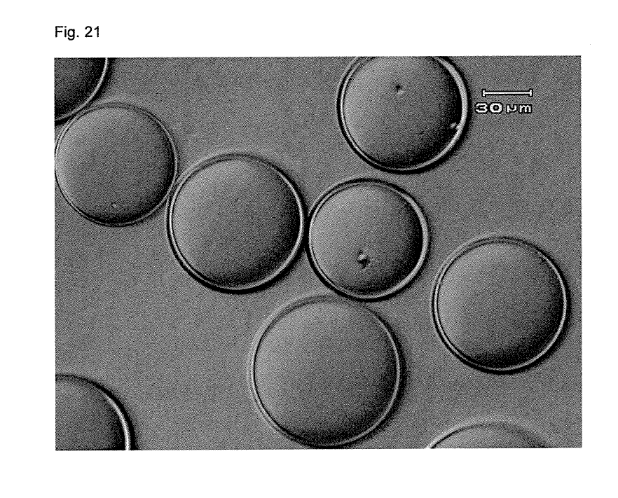

Once a population of microcapsules has been formed, the volume distribution can be narrowed by mechanical sieving through meshes of varying grades corresponding to the cross-sectional area of the openings (FIG. 20). Setting a lower volume bound greater than the largest solid polymer microsphere and smaller than the mean fluid-core microcapsule allows for the separation of the microspheres from microcapsules. By imposing upper and lower bounds through filtration or mechanical sieving allows for homogenization of the volume of a population of microcapsules.

To further homogenize the composition of a population of microcapsules, as is desirable in certain embodiments of the invention, density separation techniques can be employed. Since in most embodiments of the invention the encapsulated media has a distinct density from that of the polymer shell, and since density is linearly related to volume, solutions of varying densities can be used to separate populations of microcapsules by their volume fractions, FIG. 21.

In combination, once a population of microcapsules has been manufactured, size and volume fraction limits can be imposed during post-processing. For example, if a population of microspheres ranging between about 10 to about 250 microns in diameter containing greater than about 75 percent of the initial aqueous phase is desired, the population could be sieved through gratings with the appropriate upper and lower size bounds and then centrifuged in a sucrose solution matched to the weighted average density of about 75 percent fluid capsule. At final, collecting the microcapsules that have been sieved and that sink in the appropriate density sucrose solution would produce the desired population of microcapsules. Homogenization of the composition of the microcapsules through post-processing allows for the separation of populations of microcapsules into sub-populations with well-defined limits for mechanical rupture.

### Manufacturing Process Scaling

To increase the scale of production of the microcapsules manufactured by the methods set forth in the present invention, the ratios of the various components in the formulations have been showed to scale linearly with volume. In increasing production by an order of magnitude resulted in batches within about equivalent percent yields. Microcapsule populations will vary within an expected range of acceptable parameters due to changes in mixing container geometry and/or stirring apparatus employed. Mixing times and rates do not scale with batch size and should remain within the range of small-scale batches when scaling up to larger batches depending again upon the physical properties of the mixing setup. Additionally, all materials other than the encapsulated media and the polymer shell material, which irreversibly cures during processing, are recoverable.

### Encapsulated Agents

Oral Care Agents. Hydrogen peroxide, carbamide peroxide, methyl paraben, ethyl paraben, propyl paraben sodium salt, cetyl pyridinium chloride, sodium percarbonate, triclosan, thymol, and menthol.

Skin Care Agents. Benzoyl peroxide, salicylic acid, cetyl pyridinium chloride, quinones, azeleic acid, hydrogen peroxide, clyndamycin, erythromycin, tetracycline, minocycline, doxycyclin, retenoids (e.g. isotretinoin), mineral oil, soybean oil, and vegetable extracts and oils.

Flavored and Scented Agents. Menthol, sorbitol, cyclodextrins, soybean oil, glutamic acid salts, glycine salts, guanylic acid salts, inosinic acid salts, 5′-ribonucleotides salts, acetic acid, citric acid, malic acid, tartaric acid, iso-Amyl acetate, eugenol acetaldehyde, cinnamic aldehyde, ethyl propionate, limonene, ethyl-(E,Z)-2,4-decanoate, allyl hexanoate, benzaldehyde, ethyl-2-methyl butryrate, hexenyl trans-2-hexenal acetaldehyde, diacetyl, dimethyl sulphide, delta-deca lactone, butyric acid, dimethyl disulphide, 2-propenyl iso-thiocyanate, citral, 1-octen-3-one, terpenes (alpha pinene, beta ocimene), cis-3-hexanol, undecalactone, 2-ethyl-3-methoxy pyrazine, methional, fuaneol, cis-3-hexenol, ethyl butyrate, ethyl methyl paraben, glycidate, methyl cinnimate, 1-p-hydroxyphenyl-3-butanone, cis-3-hexenol, damascenone, alpha ionones, beta ionones, methyl-n-methyl anthranilate, thymol, trans-2-hexenal, cis-3-hexenal, 2-iso-butylthiazone, and propylene glycol.

Therapeutic Agents. Hormones and Hormone Modifying Agents: estrogen; estradiols; progesterone; progestins; follicle stimulating hormone; testosterone; leutenizing hormone releasing hormone; salmon calcitonin; human grown hormone; propanamide; prostaglandins; leukotrienes; prostacyclin; acetyl-D-3-(2′-naphtyl)-alanine-D-4-chlorophenylalanine-D-3-(3′-pyridyl)-alanine-L-serine-L-tyrosine-D-citruline-L-leucine-L-arginine-L-proline--alanine-amide; N-[4-cyano-3-(trifluoromethyl)phenyl]-3-[(4-fluorophenyl)sulfonyl]-2-hydroxy-2-methyl-(+−); erythropoietin; ghrelin; parathyroid hormone, thyroid stimulating hormone; thyroid releasing hormone; cortisol; 1-[(6-allylergolin-8β-yl)-carbonyl]-1-[3-(dimethylamino)propyl]-3-ethylurea. Chemotherapeutics: 5β,20-Epoxy-1,2α,4,7β,10β,13α-hexahydroxytax-11-en-9-one 4,10-diacetate 2-benzoate 13-ester with (2R,3S)-N-benzoyl-3-phenylisoserine; cisplatin; (2R,3S)-N-carboxy-3-phenylisoserine,N-tert-butylester,13-ester; 5β-20-epoxy-1,2α,4,7β,10β,13α-hexahydroxytax-11-en-9-one 4-acetate 2-benzoate, trihydrate; acetyl-D-β-naphthylalanyl-D-4-chlorophenylalanyl-D-3-pyridylalanyl-L-seryl-L-N-methyl-tyrosyl-D-asparayl-L-leucyl-L-N(ε)-isopropyl-lysyl-L-prolyl-D-alanyl-amide. Vitamins: A; D; E; and K.

### Encapsulants

Encapsulants provided for in this invention relate to the class of cross-linkable elastomeric polymers. In certain specific embodiments of the invention encapsulant refers but is not limited to: organosiloxanes, polyurethanes, polyisoprenes, and polybutadienes.

### Encapsulation of Active Ingredients

In one specific embodiment, the present invention provides a means of encapsulating aqueous solutions of hydrogen peroxide in organosiloxane elastomeric microcapsules for incorporation into dentifrices and chewing gums (FIG. 1). In this embodiment, a room temperature vulcanizing poly(dimethyl sixoxane) pre-polymer, a vulcanizing agent, and a suitable organic solvent (e.g., methylene chloride) comprise the encapsulant phase. The organic solvent is chosen to be immiscible with the encapsulated aqueous hydrogen peroxide phase. The encapsulated phase is suspended in the encapsulant phase in the presence of an emulsion stabilizer (e.g., poly(vinyl alcohol)), which is in turn suspended in a second aqueous phase. During the stirring of the final aqueous phase, the solvent leaves the shells of the newly formed capsules via solvent evaporation leaving the pre-polymer and vulcanizing agent to react forming a thermoset poly(dimethyl siloxane) shell around individual or groups of suspended droplets of the encapsulated media. Upon vulcanization, the resultant microcapsule population can, for example, be directly incorporated into any dentifrice or chewing gum to impart antimicrobial and tooth whitening properties.

Since many organosiloxanes are resistant to oxidation, water impermeable, pigmentable, non-caloric, and have no taste or odor, they act as excellent barriers between the dentifrice and the hydrogen peroxide while leaving the look, taste, smell, and viscosity of the dentifrice unaltered. Only upon receiving the mechanical stress imparted by tooth brushing will the capsules rupture (FIG. 2), releasing the hydrogen peroxide, which will act as both a tooth whitening and antimicrobial agent. In the case of chewing gums, mastication of the gum containing the microcapsules provides the release mechanism producing similar desired effects.

In another embodiment, the invention provides a means of encapsulating aqueous solutions or suspensions of benzoyl peroxide or salicylic acid in organosiloxane rubber for use in skin care applications such as acne-treating facial washes. During storage in the final formulation, the microcapsule walls serve as physical barriers between the base and the acne fighting ingredients until such time that they are applied to the skin when they are ruptured by the mechanical stresses imparted by the processes of lathering and scrubbing.

Another embodiment of the invention provides for the microencapsulation of solutions or suspensions of flavoring agents (i.e., menthol) for delivery in dentifrices or chewing gums. In certain embodiments water-soluble flavorants are dissolved in aqueous media or suspended in solid form in oil. In other embodiments oil-soluble flavorants are dissolved in apolar oil or suspended in water. Apolar oil encapsulation allows for the microencapsulation of oleophilic flavorants, oral hygiene, and skin care products such as menthol, triclosan, and quinones respectively. In these embodiments, often the pre-polymer and vulcanizing agent serve to alter the miscibility properties of the carrier solvent imparting immiscibility with certain apolar oils. In these embodiments, the carrier solvent modifies the viscosity of the encapsulant phase and provides a means of producing populations of oil-containing microcapsules with controllable wall thicknesses.

Studies have demonstrated that certain therapeutic agents can slowly permeate elastomeric (e.g., poly(dimethyl siloxane)) membranes (Langer & Wise Medical Applications of Controlled Release Volume I, 1984). In specific embodiments of the invention, hydrophobic therapeutic agents including steroid hormones, neurohormones, and chemotherapeutics can be encapsulated within chemically cross-linked elastomeric microcapsules to achieve prolonged pharmacokinetics. Populations of chemically cross-linked elastomeric microcapsules provide a reservoir-type delivery demonstrated for a model active for over twelve months. Pharmacokinetics of the encapsulated active is controlled by varying the shell thickness among and within populations of microcapsules, and agent permeability is also controlled by materials selection respectively. By increasing the shell thickness or utilizing a shell material of reduced permeability the release rate is slowed. Additionally, due to the ability to encapsulate a wide range of media ranging from aqueous solutions to apolar oils, the partition coefficient of the therapeutic agent can be tailored to alter release kinetics. The “partition coefficient” refers to the solubility ratio of the therapeutic agent within the elastomeric shell to the encapsulated fluid, whereby an increase in the ratio indicates an increased rate of release. In specific embodiments in which the therapeutic agent has a low solubility within the encapsulated fluid, solid-particle dispersions can also be encapsulated within chemically cross-linked polymeric microcapsules. In these embodiments the percent loading of the solid particles will affect the duration of release. As the therapeutic diffuses through the shells of the microcapsules, the solid-particulate dissolves. Therefore, increasing the solid-particle loading will increase the duration of release. The partition coefficient between the elastomeric shell and the ambient as well as the effective stir rate is determined by the physiology of site of application. In certain embodiments, suspensions of chemically cross-linked polymeric microcapsules can be delivered by intramuscular, intraperitoneal, subcutaneous, or intratumoral injection. In other embodiments, dried populations of chemically cross-linked polymeric microcapsules can be implanted intratumorally, intramuscularly, and subdermally to achieve the desired delivery. Additionally, chemically cross-linked elastomeric microcapsules containing shell-permeable actives may be incorporated into artificial tissue and organ constructs.

In the above mentioned embodiments, accurate control over formulating mechanical properties of the microcapsules, in particular having close control over the range of rupture strengths and active agent permeability within a population of microcapsules allows for a single or multiple mechanical cues to rupture each population of microcapsules, as well as differing release rates producing the desired release kinetics in response to the expected mechanical stimuli or physiological environment, respectively.

### Specific Applications

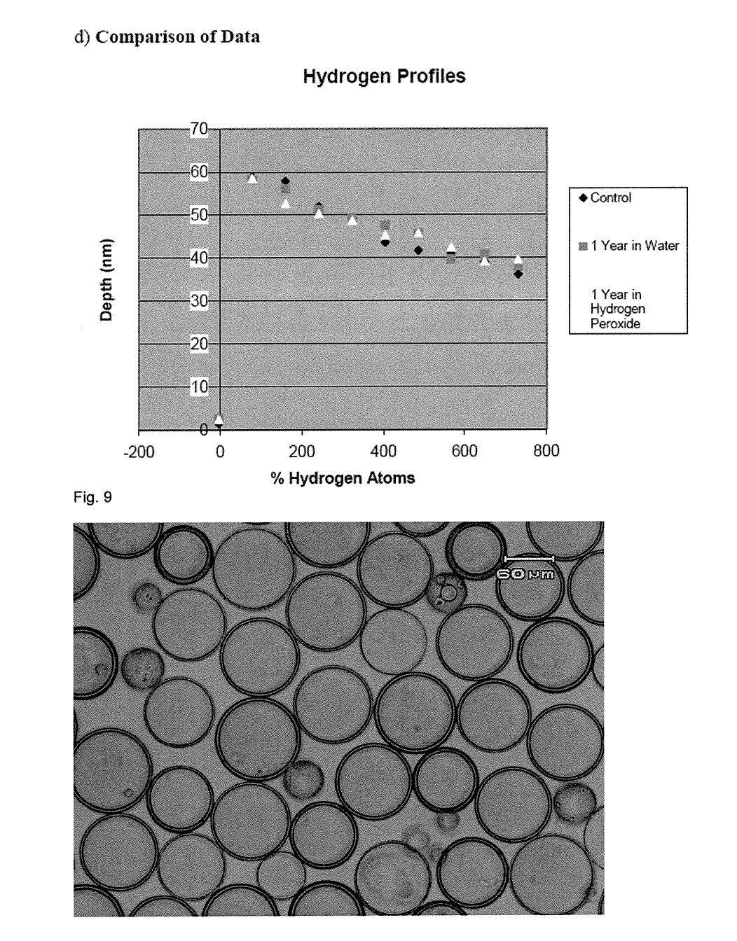

Corrosive and Reactive Hydrophilic Agent Microencapsulation. One embodiment of the invention allows for the incorporation of hydrogen peroxide either alone or in a stabilized aqueous solution into dentifrices or chewing gums, FIG. 9. In this embodiment, the initial aqueous phase is composed of the hydrogen peroxide solution with 0.1 weight percent polyvinyl alcohol as the emulsion stabilizer. In particular, a highly thermally stable aqueous hydrogen peroxide solution, Solvay Chemicals Ultra-Cosmetic Grade Hydrogen Peroxide, was mixed in the ratio of ten times the volume to a 1 weight percent solution of Celanese Celvol 205S polyvinyl alcohol to form the initial aqueous phase. The initial aqueous phase is then added to the hydrophobic phase of organosiloxane elastomer in a solution of methylene chloride in the volume ratio of five parts encapsulant phase to two parts encapsulated phase. The hydrophobic phase consisted of a mixture of the Dow Coming Sylgard 184 silicone rubber pre-polymer and cross-linking agent in a mass ratio of five to one in solution with five and one quarter times the mass of methylene chloride that has been degassed by centrifugation or sonication. Both phases were vortex mixed at a rate of 3,000 revolutions per minute for 60 seconds to form the first water-in-oil emulsion. Since Sylgard 184 is a room temperature vulcanizing organosiloxane rubber, the entire process is carried out at room temperature so as to minimize the decomposition of the hydrogen peroxide being encapsulated. Temperature can be increased or decreased to increase or decrease the rate of curing respectively, while also affecting the dissociation of the hydrogen peroxide experienced during processing.

Next five times the volume of the water-in-oil emulsion of a 1 percent solution of Celvol 205S was added before vortex mixing again for 30 seconds at 3,000 revolutions per minute. Upon completion of the vortex mixing, the contents were added to at least ten times the volume of 0.25 weight percent Celvol 205S aqueous solution stirring at a rate of 1,000 revolutions per minute, which continued until the methylene chloride has left via solvent evaporation and the silicone rubber shell has cured. The resultant microcapsules were then filtered through 70 and 100 micron Becton Dickenson Cell Strainers and centrifuged in a 1.08 grams per milliliter aqueous sucrose solution to homogenize the products. Upon completion, the microcapsules are washed with distilled water until the polyvinyl alcohol in the final aqueous solution has been diluted to a sufficiently low weight percent (e.g. less than 0.01 weight percent). At final populations of microcapsules prepared in the above manner yielded 60-70 percent encapsulation of the initial volume of hydrogen peroxide as determined by the titration method described by the United States Pharmacopeias. Populations of microcapsules made in the above manner can be incorporated into dentifrice or gum formulations to add both whitening and antimicrobial properties.

Using the discussed methods for varying mechanical properties, the above manufacturing process can be altered to achieve a population of microcapsules within a desired size range, containing a specific volume fraction of hydrogen peroxide that will rupture only when exposed to a particular set of mechanical stresses. Additionally, any aqueous solution of an active agent (i.e. an aqueous solution of the sodium salt of propyl paraben, an aqueous solution or suspension of benzoyl peroxide, etc.) may be encapsulated using the above conditions provided that the active agent does not significantly affect the viscosity of the resultant solution or the interfacial tension between the polymer containing and active containing phases.

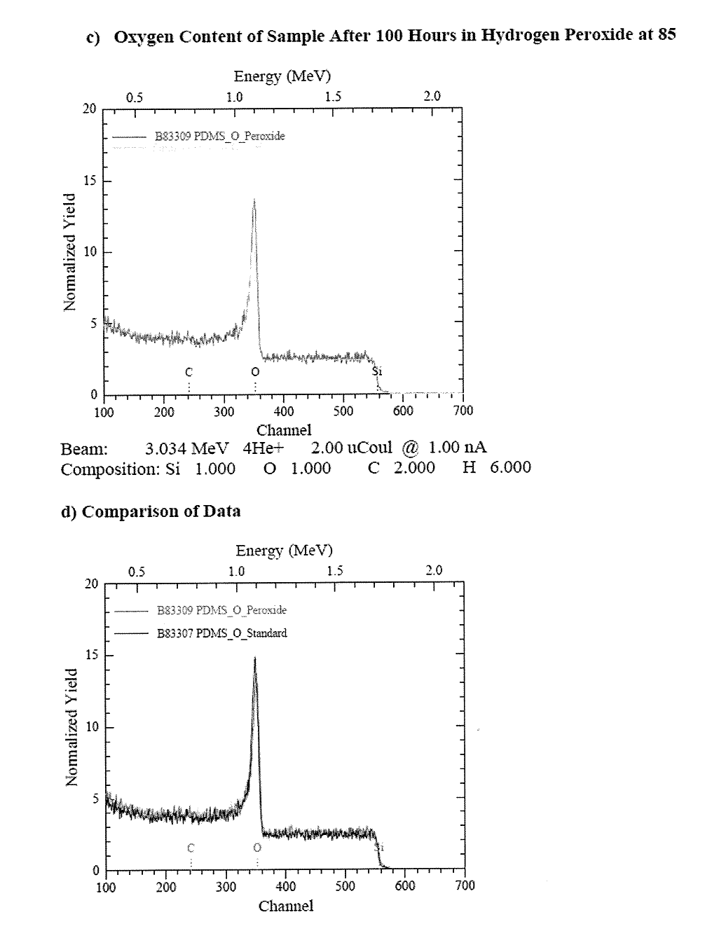

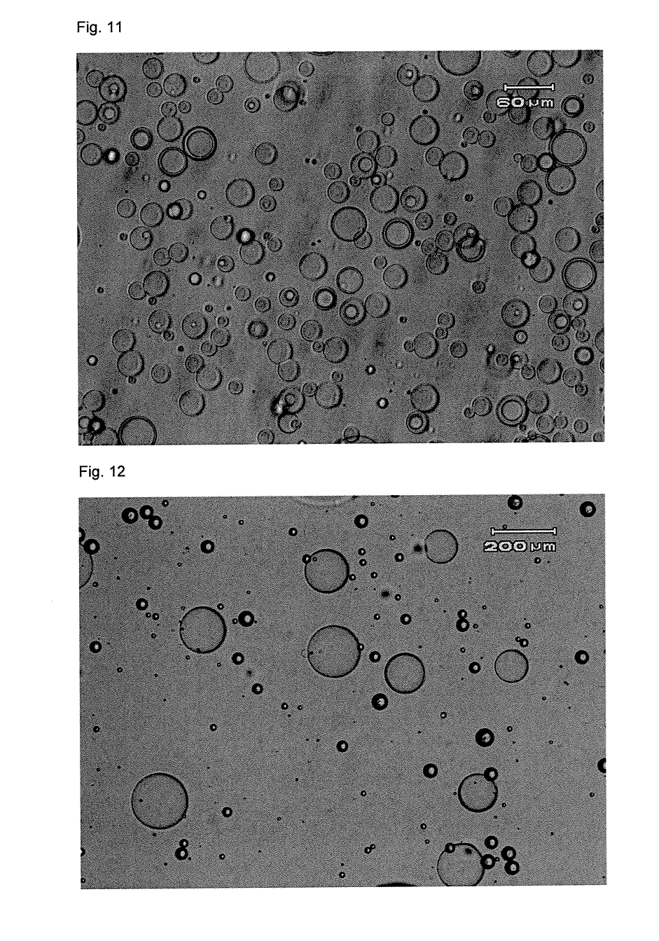

Surface Active Agent Microencapsulation. In specific embodiments of the invention in which the active agent significantly alters the interfacial tension between the encapsulant and active agent containing phases, such as in the case of an aqueous solution of cetyl pyridinium chloride, the viscosity of the encapsulated phase may also be altered to achieve the desired population of microspheres. Any agent that decreases the interfacial tension, such as cetyl pyridinium chloride due to its amphipathic nature will result in the formation of a population of smaller microcapsules on average than ones with higher interfacial tensions under the same mixing conditions, FIGS. 11 and 12. Through the addition of a chemically inert, similarly soluble agent or combination of agents to the encapsulated phase (i.e. 70 weight percent aqueous sorbitol can be added in equal volume to 10 weight percent aqueous cetyl pyridinium chloride to increase the viscosity of the encapsulated phase) resulting in a population of microcapsules with a greater mean diameter than a less viscous solution. In this way, viscosity and interfacial tension may be controlled through the addition or depletion of surfactants and thickening agents respectively to produce a population of microcapsules with a particular mean diameter.

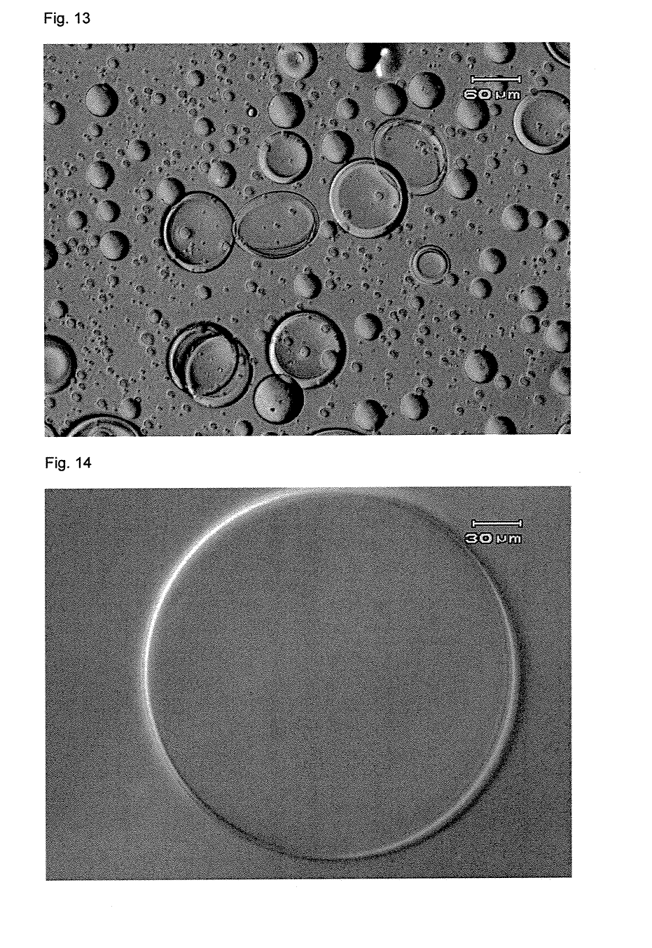

Microencapsulation of Sorbitol as an Active Agent for Controlling the Osmotic Pressure Gradient and Encapsulated Phase Rheological Properties. In another specific embodiment of the invention, an aqueous solution of sorbitol may be encapsulated by combining an aqueous sorbitol solution with the active agent solution in the above procedure. In addition to increasing the viscosity of the encapsulated phase, encapsulating sorbitol in the presence of active agents greatly increases the osmolarity of the encapsulated phase. A difference in the osmolarity of the encapsulated media and of the ambient leads to an osmotic pressure gradient across the polymer shells of the microcapsules acting in the direction of decreasing osmolarity. In the case where there is a sufficiently great osmotic pressure gradient creating a pressure gradient acting normal to the capsule surface inward towards the center of the microcapsule, dimpling of the spherical shell is observed in proportion to the magnitude of the gradient, FIG. 13. Increasing the osmolarity of the encapsulated media decreases the magnitude or reverses the direction of the osmotic pressure gradient ensuring the spherical morphology of the capsule shells in the ambient environment, FIG. 14, as is desirable in certain embodiments of the invention.

Additionally, agents such as sorbitol can be added to the aqueous encapsulated phases to increase its viscosity. By increasing the viscosity of the encapsulated phase larger droplets of the encapsulated phase will form in the encapsulant phase resulting in populations of microcapsules with larger mean diameters than those with lower encapsulated phase viscosities given the same mechanical stirring conditions.

Apolar Oil Microencapsulation. Certain mixtures of carrier solvent, pre-polymer, and curing agent allow for phase separation between the multi-component encapsulant phase and apolar oils. In one such embodiment, a ten to one weight ratio of Dow-Coming Sylgard 184 pre-polymer to curing agent is dissolved in a volume ratio of two parts in five parts of methylene chloride. Apolar oil (e.g., Johnson's Baby Oil, a low viscosity, orally acceptable mineral oil) is added to the multi-component encapsulant phase in the volume ratio of one part in five respectively and the phase-separated mixture is emulsified by vortex mixing for 30 seconds at 3,000 revolutions per minute. The resultant emulsion is then added to at least ten times the volume of 0.25 weight percent Celvol 205s aqueous solution stirring at a rate of 1,000 revolutions per minute. The resultant population of microcapsules (FIG. 16) is comprised of elastomeric shells surrounding oil cores for incorporation of flavored or scented oils into any number of formulations including aqueous-based products.

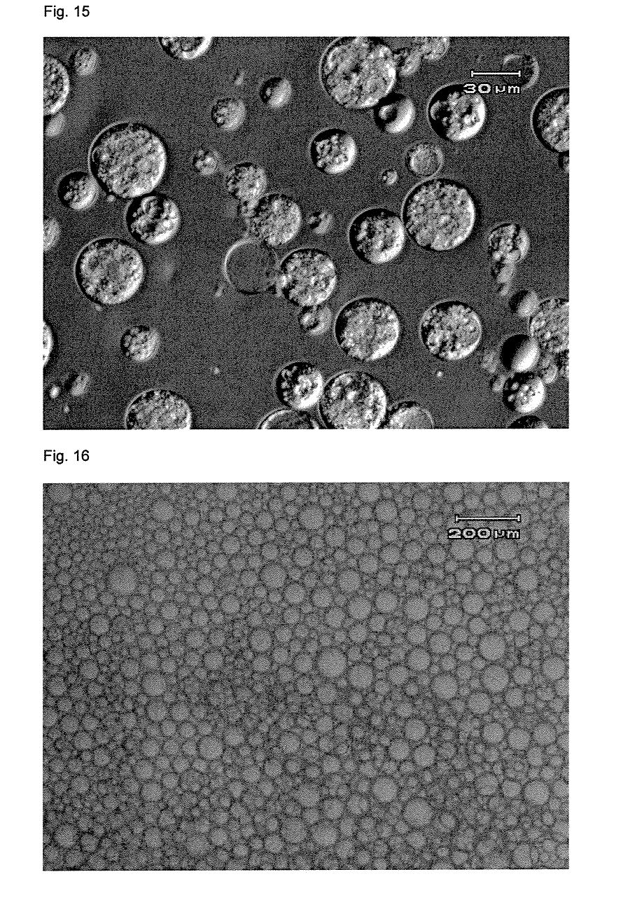

Solid Microparticle Suspension in Apolar Oil Microencapsulation. In other embodiments of the invention, water-soluble, or water-labile, active agents can be suspended in solid form in an apolar oil (e.g., light mineral oil), which is immiscible with Sylgard 184 pre-polymer and curing agent due primarily to differences in polarity and can therefore be microencapsulated using the above method having substituted the aqueous encapsulated phase with the oleophilic suspension. In this way, water labile active agents (i.e., sodium percarbonate), and solid-form active agents (i.e., menthol crystals) may be encapsulated in mechanically-ruptured polymer microcapsules provided that no dimension of the solid particles exceeds the diameter of the lumen of the microcapsule (FIG. 15).

Thermo-Mechanical Microcapsule Rupture. In addition to rupture by mechanical force, chemically cross-linked elastomeric microcapsules can be ruptured thermo-mechanically. In specific embodiments in which chemically cross-linked elastomeric microcapsules contain agents micro-wave responsive agents (e.g., water), micro-wave radiation can be utilized to rupture the capsule walls. To demonstrate proof of principle, water-core elastomeric microcapsules produced by the above methods, were stored for six months at room temperature, and were then filtered from aqueous suspension after curing using a Falcon 40-micron Cell Strainer. Once separated from the aqueous phase, the microcapsules were rinsed with ethanol to remove water and poly(vinyl alcohol) from the outer surface of the microcapsules. Microcapsules were then introduced into food grade soybean oil and were heated in a standard Goldstar microwave oven for thirty seconds. After being exposed to micro-wave radiation, nearly all chemically-cross-linked elastomeric microcapsules had ruptured, releasing their contents, as shown in FIG. 17. Experimentation determined that rapid thermal expansion of the water vapor within the microcapsules caused a pressure gradient across the capsule shell sufficient to rupture the shell. Such thermo-mechanically ruptured microcapsules can be used to release thermally-stable flavoring and scented agents (e.g. flavored oils) upon microwaving for a given period of time. Additionally, thermo-mechanical rupture by heating may be desirable in dryer sheet applications.

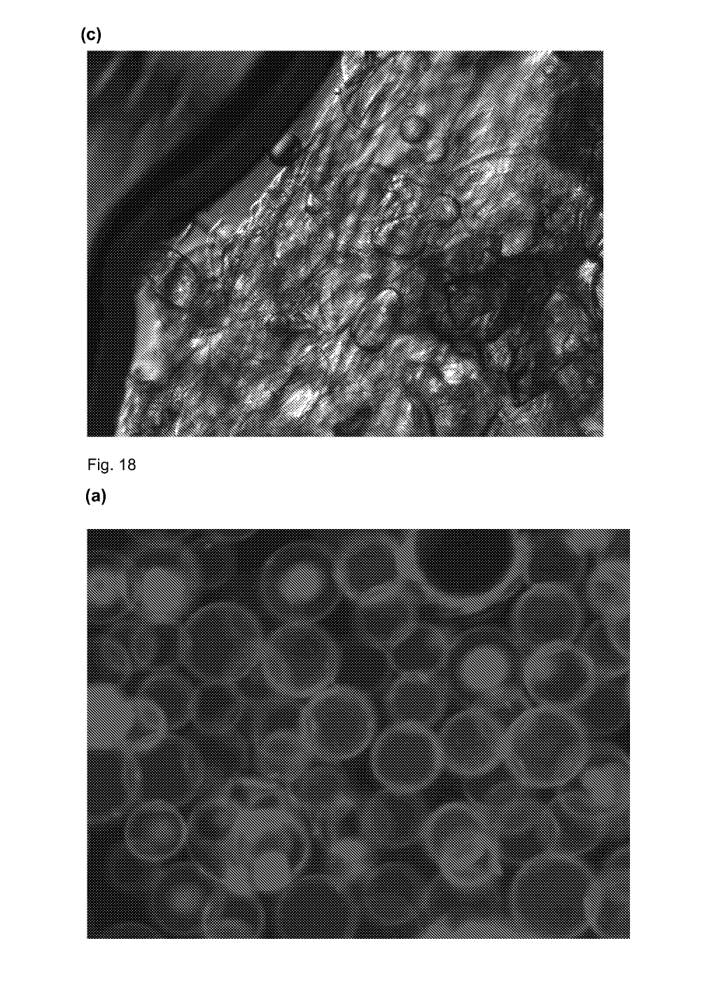

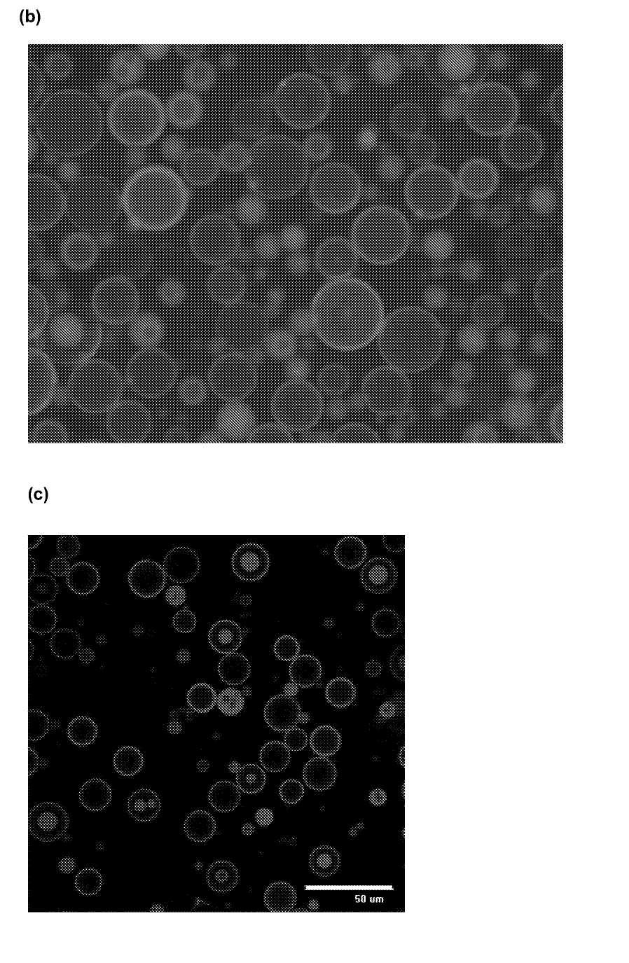

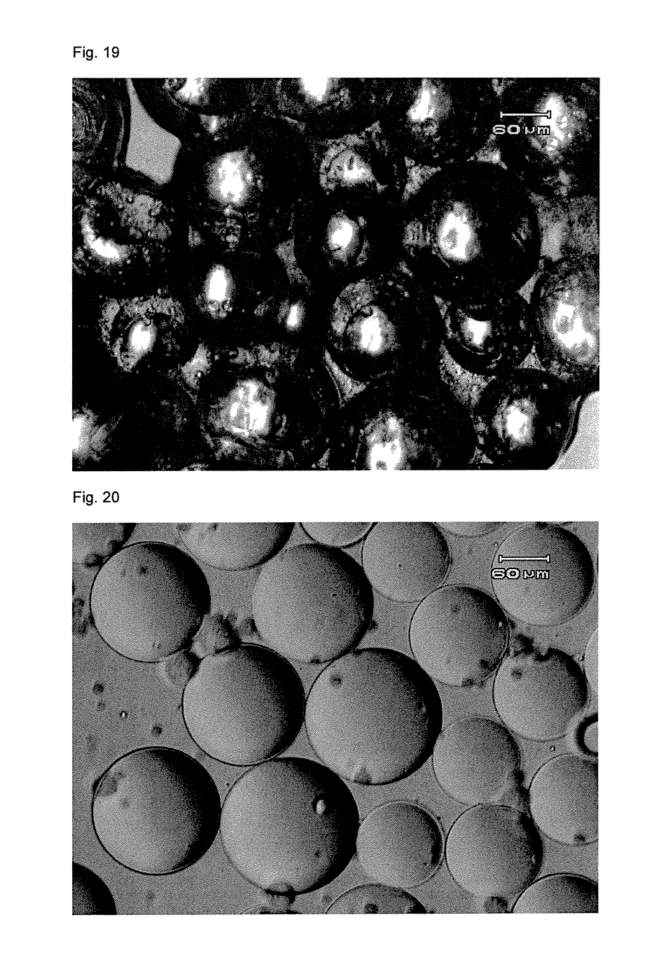

Elastomer-Permeable Therapeutic Agent Microencapsulation. Rhodamine dye was encapsulated within chemically cross-linked poly(dimethyl siloxane) microcapsules utilizing the hydrophilic agent encapsulation methods at low concentrations due to the hydrophilicity of the aqueous solution. However, rhodamine is hydrophobic and permeates vulcanized poly(dimethyl siloxane) in the same fashion as a hydrophobic therapeutic agent. The resultant population of microspheres was sieved and re-suspended in a dilute aqueous poly(vinyl alcohol) solution. Upon resuspension in the rhodamine-free aqueous solution, a series of fluorescence photomicrographs were obtained. One representative photomicrograph is shown in FIG. 18 demonstrating the lack of detectable quantities of rhodamine in the surrounding media initially. No burst effect was observed as expected with a reservoir-type system without an active-saturated shell. Another representative fluorescence photomicrograph obtained more than one year after fabrication, shown in FIG. 18, visually demonstrates an increased ambient concentration of rhodamine dye and decreased rhodamine concentration within the microcapsules evidencing extended release of a model therapeutic for greater than one year unstirred at room temperature.

To further investigate the shell-permeability and partition coefficient of the rhodamine within chemically cross-linked elastomeric microcapsules samples of the population of microcapsules that was tested for release were imaged by confocal microscopy. Photomicrographs demonstrate that the rhodamine, which was initially undetectable within the capsule shells, enters the poly(dimethyl siloxane) shell in this embodiment as shown in FIG. 18.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed is:

1. A population of microcapsules comprising:
(a) chemically cross-linked elastomeric shells; and
(b) an encapsulated phase comprising active agents.

2. The population of microcapsules in claim 1, wherein the encapsulated phase contains active agents in aqueous solution cores.

3. The population of microcapsules of claim 2, wherein the encapsulated phase contains water-immiscible fluid droplets

4. The population of microcapsules of claim 2, wherein the encapsulated phase contains water-insoluble solid particles.

5. The population of microcapsules in claim 1, wherein the encapsulated phase contains apolar oil cores.

6. The population of microcapsules of claim 5, wherein the apolar oil contains suspended droplets of aqueous solutions.

7. The population of microcapsules of claim 5, wherein the apolar oil contains apolar-oil-immiscible solid particles.

8. The population of microcapsules of claim 7, wherein the solid particles are water soluble or water labile.

9. The population of microcapsules in claim 1, wherein each microcapsule comprises a single distinct wall and a single core.

10. The population of microcapsules in claim 1, wherein each microcapsule comprises multiple, distinct cores.

11. The population of microcapsules in claim 1, wherein the mean diameter of the microcapsules ranges from about 2.5 to about 2,500 microns.

12. The population of microcapsules in claim 1, wherein the encapsulated active agent is shell-permeable.

13. The population of microcapsules of claim 12, wherein the shell-permeable active agent is released from the chemically cross-linked elastomeric microcapsules for at least one month.

14. The population of microcapsules of claim 12, wherein the shell-permeable active is released from the chemically cross-linked elastomeric microcapsules for at least three months.

15. The population of microcapsules of claim 12, wherein the shell-permeable active is released from the chemically cross-linked elastomeric microcapsules for at least six months.

16. The population of microcapsules of claim 12, wherein the shell-permeable active is released from the chemically cross-linked elastomeric microcapsules for at least one year.

17. The population of microcapsules of claim 1, wherein the chemically cross-linked elastomer shell is poly(dimethyl siloxane).

18. The populations of microcapsules of claim 1, wherein a carrier solvent is utilized to manufacture populations of microcapsules having a mean shell thicknesses of fewer than about 50 microns.

19. The population of microcapsules of claim 1, wherein a carrier solvent is utilized to manufacture populations of microcapsules having a mean shell thickness of fewer than about 5 microns.

20. The population of microcapsules of claim 1, wherein a carrier solvent is utilized to manufacture populations of microcapsules having a mean shell thickness of fewer than about 2 microns.

21. The population of microcapsules of claim 2, wherein the active agent is an aqueous solution of hydrogen peroxide.

22. The population of microcapsules of claim 1, wherein the microcapsules are incorporated into a dentifrice as a tooth whitening and antimicrobial agent.

23. The population of microcapsules of claim 1, wherein the microcapsules are incorporated into a chewing gum as a tooth whitening and antimicrobial agent.

24. The population of microcapsules of claim 1, wherein the shell ruptures, thereby releasing the encapsulated agents, by one or more methods selected from the group consisting of exposure to microwave radiation, thermal expansion of the encapsulated agent, mechanical stresses imparted by tooth brushing, mechanical stresses imparted by mastication, and mechanical stresses imparted by lathering or scrubbing.

25. The population of microcapsules of claim 1, wherein the microcapsules comprise therapeutic agents for delivery by injection or implantation.

26. The population of microcapsules of claim 1, wherein the active agents are selected from the groups consisting of chemotherapeutic agents, hormones or hormone modifying agents, and vitamins.

27. A method of forming a population of microcapsules, in which the method comprises emulsifying the encapsulated phase within a dual-component encapsulant phase comprising a pre-polymer and curing agent, and emulsifying both phases within a third, aqueous phase.

28. A method of forming a population of microcapsules, in which the method comprises emulsifying the encapsulated phase within a multi-component encapsulant phase comprising a pre-polymer, a curing agent, and a carrier solvent, and emulsifying both phases within a third, aqueous phase, enabling carrier solvent evaporation.

29. A method of forming a population of microcapsules, in which thermally-sensitive active agents are encapsulated at or below about 30° C. utilizing a room temperature vulcanizing pre-polymer and curing agent.

30. A method of forming a population of microcapsules, in which the pre-polymer and curing agents are cured at a temperature greater than about 30 but less than about 100° C.

31. A method for producing chemically cross-linked elastomeric microcapsules containing a high osmolarity solution, wherein the high osmolarity is achieved by encapsulating an inactive agent within the encapsulated phases in addition to the active agent, wherein the inactive agent is selected from the group consisting of osmolarity-increasing, viscosity-increasing, and surface-active agents.
